SEQUENCE LISTING (8 sequences)

SEQ ID NO: 1 - DNA, 375 bp, Streptoalloteichus hindustanus
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 2 - DNA, 1026 bp, Unknown
  Hygromycin resistance gene
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 3 - DNA, 795 bp, Unknown
  Neomycin resistance gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 4 - DNA, 23 bp, Artificial
  Forward primer
accagtgccgttccggtgctcac

SEQ ID NO: 5 - DNA, 22 bp, Artificial
  Reverse primer
tgctcgccgatctcggtcatgg

SEQ ID NO: 6 - DNA, 37 bp, Artificial
  (dT)17-AP primer
ggccacgcgtcgactagtacttttttttttttttttt

SEQ ID NO: 7 - DNA, 828 bp, Unknown
  Neomycin resistance gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttcactagttacccatacgacgtcccagactacgcttga

SEQ ID NO: 8 - DNA, 1026 bp, Unknown; identical to SEQ ID NO: 2
  Hygromycin resistance gene

(19) United States
(12) Patent Application Publication
(10) Pub. No.: US 2015/0368655 A1
(43) Pub. Date: Dec. 24, 2015

(54) TRANSFORMED EUGLENA AND PROCESS FOR PRODUCING SAME

(71) Applicants: OSAKA PREFECTURE UNIVERSITY PUBLIC CORPORATION, Osaka (JP); euglena Co., Ltd., Tokyo (JP)

(72) Inventors: Masami Nakazawa, Osaka (JP); Daiki Haruguchi, Osaka (JP); Mitsuhiro Ueda, Osaka (JP); Kazutaka Miyatake, Osaka (JP)

(73) Assignees: Osaka Prefecture University Public Corporation, Osaka (JP); euglena Co., Ltd., Tokyo (JP)

(21) Appl. No.: 14/655,228

(22) PCT Filed: Dec. 25, 2013
(86) PCT No.: PCT/JP2013/084618

§ 371 Date: Jun. 24, 2015

(30) Foreign Application Priority Data
Dec. 26, 2012 (JP) 2012-283531

(51) Int. Cl. C12N 15/79

(52) U.S. Cl. CPC C12N 15/79

(57) ABSTRACT

A Euglena carrying a foreign gene of interest in an expressible manner is provided.

A Euglena carrying a drug resistance gene and a foreign gene of interest in an expressible manner.

### TECHNICAL FIELD

The present invention relates to transformed Euglena and a method for producing transformed Euglena.

### BACKGROUND ART

Euglena is a protozoan classified into both the Kingdom Animalia and the Kingdom Plantae because it has the ability to grow autotrophically through photosynthesis in chloroplasts as well as having motility with fragella. Euglena does not have a cell wall in its cell structure, but is characterized in that it is covered with soft tissue containing a protein called pellicle as a main component.

Euglena, which has high capacity to absorb carbon dioxide, exhibits good growth even in the presence of carbon dioxide at a very high concentration of 40% by performing photosynthesis. Under anaerobic conditions, Euglena produces wax esters by fermentation from β1,3-glucan paramylon, which is a storage polysaccharide. The wax esters can be easily converted to biodiesel. Thus, Euglena can be described as an organism that enables fuel production along with reducing carbon dioxide.

Industrial application of Euglena has also been proposed in various other fields.

A variety of attempts have been made to introduce nucleic acid into Euglena.

For example, successes in introducing double-stranded RNA into Euglena by electroporation and allowing specific mRNA to disappear by RNAi have been reported (Non-patent Literature 1 and 2). Additionally, various attempts have been made to transform Euglena.

### CITATION LIST

### Non-patent Literature

NPL 1: Iseki, M. et al., “A blue-light-activated adenylyl cyclase mediates photoavoidance in Euglena gracilis,” Nature, 2002, 415
NPL 2: Ishikawa, T. et al., “Euglena fracilis ascorbate peroxidase forms an intramolecular dimeric structure: its unique molecular characterization,” Biochemical Journal, 426, pp. 125-134

### SUMMARY OF INVENTION

### Technical Problem

An object of the present invention is to transform Euglena. More specifically, an object of the present invention is to provide transformed Euglena and a method for transforming Euglena.

### Solution to Problem

The present inventors found that previously attempted methods for transforming Euglena are problematic in that the introduced gene cannot be stably retained. To solve this problem, the inventors conducted extensive research. First, based on a report of double-stranded RNA being introduced into Euglena to induce RNAi, the inventors investigated whether transformation can be performed in the same manner. More specifically, they attempted to introduce genes by electroporation. However, no Euglena carrying the introduced genes in an expressible manner was obtained.

The inventors then conceived of transforming Euglena by using the Agrobacterium method as a completely different process, and it has become evident that Euglena carrying the introduced genes in an expressible manner can be actually obtained by the method.

The inventors further conducted extensive research based on the above finding and accomplished the present invention. The invention is described below.

### 1. Euglena of the Present Invention

[1-1]

A Euglena carrying a drug resistance gene and a foreign gene of interest in an expressible manner.

[1-2]

The Euglena according to [1-1], which carries the drug resistance gene and the foreign gene of interest in an expressible manner until it is subcultured for at least 10 passages in the absence of the drug.

[1-3]

The Euglena according to [1-1] or [1-2], wherein the drug is zeocin, hygromycin, or G418.

[1-4]

The Euglena according to any one of [1-1] to [1-3], wherein the drug resistance gene and the foreign gene of interest are under control of at least an endogenous promoter in the Euglena.

[1-5]

The Euglena according to any one of [1-1] to [1-4], wherein the drug resistance gene and the foreign gene of interest are integrated in the genome.

[1-6]

The Euglena according to any one of [1-1] to [1-5], which is obtainable by a method comprising the step of (1) introducing the drug resistance gene and the foreign gene of interest into a Euglena by the Agrobacterium method.

[1-7]

The Euglena according to [1-6], which is obtainable by the method further comprising the step of (2) culturing the Euglena obtained in step (1) in the presence of the drug.

[1-8]

The Euglena according to [1-7], wherein the culture is performed at a pH of 6 to 8.

### 2. Method for Producing Euglena of the Present Invention

[2-1]

A method for producing a Euglena carrying a drug resistance gene and a foreign gene of interest in an expressible manner, the method comprising the step of:

(1) introducing the drug resistance gene and the foreign gene of interest into a Euglena by the Agrobacterium method.
[2-2]

The method according to [2-1], further comprising the step of:

(2) culturing the Euglena obtained in step (1) in the presence of the drug.
[2-3]

The method according to [2-1] or [2-2], wherein the drug is zeocin, hygromycin, or G418.

[2-4]

The method according to [2-3], wherein the culture is performed at a pH of 6 to 8.

[2-5]

The method according to any one of [2-1] to [2-4], wherein the drug resistance gene and the foreign gene of interest are under control of at least a Euglena endogenous promoter.

[2-6]

The method according to any one of [2-1] to [2-5], wherein, in the Euglena produced, the drug resistance gene and the foreign gene of interest are integrated in the chromosomal genome by homologous recombination.

### Advantageous Effects of Invention

The present invention makes it possible to provide a Euglena transformed with a gene of interest. The invention also makes it possible to provide a method for transforming a Euglena with a gene of interest. The invention enables the introduced trait to be retained for a long period of time and thus, for example, is more suitable for substance production.

### BRIEF DESCRIPTION OF DRAWINGS

FIG. 1 is a plasmid map of pCMV/Zeo.

FIG. 2 is a plasmid map of pGLuc-Basic.

FIG. 3 is a plasmid map of pBIG2RHPH2 and illustrates an inserted gene for pBIG2R/pCMV/Zeo and pBIG2R/pNOR/Zeo.

FIG. 4 illustrates conditions of PCR performed in the Examples.

FIG. 5 is a graph showing the growth of the Euglena wild strain in liquid media (zeocin: 100 μg/ml).

FIG. 6 is photographs, instead of drawings, showing the growth of the Euglena wild strain in plate media.

FIG. 7 is a graph showing the difference in the growth of the Euglena wild strain in a KH medium at each pH.

FIG. 8 is a graph showing the effect of cefotaxime on the Euglena wild strain.

FIG. 9 is a graph showing the effect of G418 on the Euglena wild strain.

FIG. 10 is a graph showing the effect of hygromycin on the Euglena wild strain.

FIG. 11 is a graph showing the growth of a Euglena transformant (zeocin concentration: μg/ml).

FIG. 12 is a photograph, instead of a drawing, showing the results of detecting an introduced gene from Euglena transformant DNA by PCR.

FIG. 13 is a photograph, instead of a drawing, showing the results of detecting a transcription product by using RT-PCR.

FIG. 14 is a graph showing the stability of an introduced trait of a Euglena transformant (drug resistance).

FIG. 15 is a graph showing the stability of an introduced trait of a Euglena transformant.

FIG. 16 is a photograph showing the results of detecting drug resistance gene DNA in a transformant into which a neomycin resistance gene was introduced.

FIG. 17 is a photograph showing the results of detecting a drug resistance gene transcription product in a transformant into which a neomycin resistance gene was introduced.

FIG. 18 is a photograph showing the results of detecting drug resistance gene DNA in a transformant into which a hygromycin resistance gene was introduced.

FIG. 19 is a photograph showing the results of detecting a transcription product of a drug resistance gene in a transformant in which a hygromycin resistance gene was introduced.

### DESCRIPTION OF EMBODIMENTS

### 1. Euglena of the Present Invention

The Euglena of the present invention carries a drug resistance gene and a foreign gene of interest in an expressible manner.

The Euglena used in the present invention is not particularly limited as long as it generally belongs to the genus Euglena taxonomically. The species of the genus Euglena is not particularly limited. Examples include Euglena gracilis, Euglena gracilis var. bacillaris, Euglena viridis, Astasia longa, and the like.

In the present invention, it is particularly preferable to use Euglena gracilis as a Euglena because, for example, (i) an axenic strain can be easily obtained, and (ii) Euglena gracilis is adaptable to both heterotrophic growth environments and autotrophic growth environments.

The drug resistance gene is not particularly limited as long as it can be a drug selection marker that is effective for Euglena. Examples include genes resistant to zeocin, hygromycin, G418, etc. The drug resistance gene is preferably a gene resistant to zeocin.

Examples of zeocin resistance genes include genes comprising the base sequence set forth in SEQ ID NO: 1 (Streptoalloteichus hindustanus bleomycin resistance gene (Sh ble)); however, the zeocin resistance gene is not particularly limited to these genes, and may be any gene as long as it is functionally equivalent to these.

Examples of hygromycin resistance genes include genes comprising the base sequence set forth in SEQ ID NO: 2 or 7; however, the hygromycin resistance gene is not particularly limited to these genes, and may be any gene as long as it is functionally equivalent to these.

Examples of G418 resistance genes include genes comprising the base sequence set forth in SEQ ID NO: 3 or 8; however, the G418 resistance gene is not particularly limited to these genes, and may be any gene as long as it is functionally equivalent to these.

The Euglena of the present invention has a drug resistance gene and thus has the advantage that even if the wild strain of Euglena is also present or if a non-transformant in which the introduced gene is lost becomes produced, it is possible to selectively grow a transformant alone.

There is no particular limitation on the foreign gene of interest. The desired foreign gene can be selected according to the intended purpose. For example, to enhance cell growth of Euglena and wax ester fermentation, a gene encoding pyruvate:NADP⁺ oxidoreductase can be selected. The amount of acetyl-CoA in mitochondria can be increased by increasing the expression amount of pyruvate:NADP⁺ oxidoreductase.

To homogenize wax esters produced by Euglena, a gene encoding 3-ketoacyl-CoA thiolase can be selected. It is possible to increase the accumulation amount of the longest acyl-CoA that can be reacted by increasing the expression amount of 3-ketoacyl-CoA thiolase.

To control the amount of carbon flowing into the TCA cycle of Euglena, a gene encoding citrate synthase can be selected. The amount of storage polysaccharide used as a starting material of wax esters in the cells can be increased by increasing the expression amount of citrate synthase.

Furthermore, to facilitate carbon metabolism in the TCA cycle of Euglena, the growth of cells can be promoted by increasing the expression amount of 2-oxoglutarate decarboxylase, which is involved in the only irreversible reaction in the TCA cycle.

In addition, a synthesis gene of a useful substance, such as human interferon, can be used as the foreign gene of interest; therefore, the Euglena of the present invention can be used as a system that produces such useful substances.

Further, a selection marker may be used as the foreign gene of interest. The selection marker can be used for the same purpose as the drug resistance gene mentioned above. More specifically, the use of the selection marker has the advantage of making it possible to selectively grow a transformant alone even if the wild strain of Euglena is also present or if a non-transformant in which the introduced gene is lost becomes produced.

The phrase “carrying a drug resistance gene and a foreign gene of interest in an expressible manner” as used herein is not particularly limited and, for example, refers to carrying the drug resistance gene and foreign gene of interest mentioned above in an expressible manner until the Euglena is subcultured for at least 10 passages in the absence of the drug to which the introduced drug resistance gene shows resistance. In this case, the number of passages in the subculturing is more preferably 15, and even more preferably 20. Whether the Euglena carries the genes in an expressible manner can be confirmed by performing RT-PCR on mRNA extracted from the Euglena, using primers that can amplify the genes.

In the present invention, the drug resistance gene and the foreign gene of interest are preferably under control of at least a Euglena endogenous promoter. Examples of Euglena endogenous promoters include, but are not particularly limited to promoters of, pyruvate:NADP⁺ oxidoreductase, glyceraldehyde-3-phosphate dehydrogenase, carbonic anhydrase, bifunctional glyoxylate cycle enzyme, α-tubulin, etc. In particular, pyruvate:NADP⁺ oxidoreductase promoter is preferable.

In the present invention, the drug resistance gene and the foreign gene of interest are preferably integrated in the genome. There is no particular limitation, but the drug resistance gene and the foreign gene of interest are preferably integrated in the genome by homologous recombination. Whether the genes are integrated in the genome can be confirmed by Southern blotting. However, the number of copies of the introduced genes may not be high enough, and no clear signal may be obtained by Southern blotting. In such a case, RT-PCR using the primers that can amplify the introduced genes can be performed on mRNA extracted from the Euglena continuously subcultured for 20 passages in the absence of the drug to which the introduced drug resistance gene shows resistance, and if the target sequences are amplified, it can be determined that the genes are integrated in the genome. More specifically, it can be determined that detection of the introduced genes by RT-PCR from the cells continuously subcultured in the absence of selective pressure indicates that there is a high probability that the introduced genes are integrated in the genome.

### 2. Method for Producing Euglena of the Present Invention

The method for producing a Euglena of the present invention is a method for producing a Euglena carrying a drug resistance gene and a foreign gene of interest in an expressible manner, the method comprising the step of:

(1) introducing the drug resistance gene and the foreign gene of interest into a Euglena by the Agrobacterium method.

The drug resistance gene, the foreign gene of interest, and the Euglena are as explained in Section 1 above.

The Agrobacterium method was originally used as a transformation method for plants. The Agrobacterium method uses Agrobacterium tumefaciens (hereafter, “Agrobacterium”), a gram-negative soil bacterium that causes tumors called crown gall in plants. A gene region called transferred-DNA (T-DNA), which is a portion of a large plasmid called tumor-inducing (Ti) plasmid possessed by Agrobacterium, is removed, enters plant cells through a type-IV secretion system, and is introduced into the nuclear genome by homologous recombination, leading to tumor formation. The removal of T-DNA requires a group of genes called the virulence (vir) region. Although the sequences of both regions are present on the Ti plasmid, these regions are known to function together even if they are not on the same plasmid.

The Agrobacterium method is preferably a binary vector method. The binary vector method, which was developed utilizing the properties of Agrobacterium described above, uses two different plasmids. In the binary vector method, the vir region and the T-DNA region, which are inherently on the same plasmid, are carried on different plasmids, and these plasmids are simultaneously used. More specifically, Agrobacterium carrying both a plasmid containing only the vir region (helper plasmid) and a plasmid containing only the T-DNA region (binary vector) is used. The process is as follows. First, a gene to be introduced into an organism used as a host is inserted into the T-DNA region on a binary vector, and this plasmid is introduced into Agrobacterium carrying a helper plasmid. The thus-obtained Agrobacterium carrying the two types of plasmids is co-cultured with the host organism, thereby introducing the desired gene into the host. The co-culturing method for Agrobacterium infection may be either a method using a solid medium or a method using a liquid medium. A method using a liquid medium is more preferable. Additionally, in co-culturing, it is desirable to add a phenol compound as an Agrobacterium infection induction substance. The phenol compound used as an infection induction substance is most desirably acetosyringone.

The method for producing a Euglena of the present invention may further comprises the step of:

(2) culturing the Euglena obtained in step (1) in the presence of the drug to which the introduced drug resistance gene shows resistance.

Conditions for the culture are not particularly limited. When zeocin or hygromycin is used as the drug, the culture is preferably performed at a pH of 6 to 8. When G418 is used as the drug, the culture is preferably performed at a pH of 5 to 8. Although a pH near 5.0 is generally considered to be advantageous to the growth of Euglena, performing the culture under the above conditions is advantageous since these drugs are more stably maintained, and a Euglena carrying a drug resistance gene and a foreign gene of interest in an expressible manner is more efficiently obtained.

When a drug that is stable to acids and bases is used as the drug, the culture conditions can be set without being particularly affected by pH conditions.

There is no particular limitation on the culture conditions in step (2). Examples include the following conditions. For example, the culture is performed in a KH selective plate medium. The concentration of the drug in the medium is not particularly limited. When a zeocin resistance gene is introduced, zeocin is used in an amount of, for example, 20 to 100 μg/ml. If necessary, one or more other drugs may be further added to the medium. There is no particular limitation, and when a zeocin resistance gene is introduced, for example, cefotaxime may be further added to the medium in an amount of 50 to 200 μg/ml. This prevents Agrobacterium alone from forming colonies after the establishment of infection, and is thus advantageous.

When a hygromycin resistance gene is introduced, hygromycin is used in an amount of, for example, 5 to 100 μg/ml. Further, cefotaxime may be added to the medium in an amount of 50 to 200 μg/ml. This prevents Agrobacterium alone from forming colonies after the establishment of infection, and is thus advantageous.

When a G418 resistance gene is introduced, G418 is used in an amount of, for example, 5 to 100 μg/ml. Further, cefotaxime may be added to the medium in an amount of 50 to 200 μg/ml. This prevents Agrobacterium alone from forming colonies after the establishment of infection, and is thus advantageous.

The number of cells at the start of culture is not particularly limited and is, for example, 1×10⁴ to 1×10⁸ cells.

The culture period is not particularly limited, and is, for example, 2 to 7 days.

Step (2) may be performed only once or, if necessary, may be performed two or more times.

When the step is performed two or more times, the number of cells at the start of culture is not particularly limited, but may be reduced in a stepwise manner if necessary. Although there is no particular limitation, the number of cells at the start of culture may be reduced to about one-fifth to about one-half of that of the previous stage.

### EXAMPLES

Examples are given below to illustrate the present invention in more detail, but it is not limited to these Examples.

### 1. Euglena

The Euglena used in the Examples is as follows.

Euglena gracilis Klebs Z strain (hereafter, “Euglena wild strain”), which has chloroplasts, was used.

Agrobacterium tumefaciens C58C1 strain (Brad, G. et al., “Genome sequence of the plant pathogen and biotechnology agent Agrobacterium tumefaciens C58,” Science, 2001, 294: 2323) (hereafter, “Agrobacterium”), which carries a helper plasmid essential for a binary vector method and is a rifampicin resistant strain, was used.

Escherichia coli DH5α strain was used as a host for amplifying constructed plasmids.

### 2. Method for Culturing Euglena

In the Examples, Euglena was cultured as described below.

Koren-Hutner medium (KH medium) was used as a heterotrophic medium (Table 1). 150 ml of the KH medium adjusted to a pH of 6.8 was placed in a 500 ml Sakaguchi flask and sterilized by autoclaving at 121° C. for 15 minutes. 1 ml of Euglena that reached the stationary phase by culturing for about 4 to 7 days (10 to 15×10⁶ cells/ml) was inoculated into the medium, followed by culture with shaking at 27° C. under continuous light conditions for 24 hours. A plate medium was prepared by adding 1.0%(w/v) of agar powder.

TABLE 1

| | Amount | | Amount |
|---|---|---|---|
| Components | (g/l) | Components | (mg/l) |
| Arginine Hydrochloride | 0.5 | Disodium EDTA | 50.0 |
| Aspartic Acid | 0.3 | Iron Ammonium Sulfate | 50.0 |
| Glucose | 12.0 | Hexahydrate | |
| Glutamic Acid | 4.0 | Manganese Sulfate | 18.0 |
| Glycine | 0.3 | Monohydrate | |
| Histidine Hydrochloride | 0.05 | Zinc Sulfate Heptahydrate | 25.0 |
| Malic Acid | 6.5 | Sodium Molybdate | 4.0 |
| Trisodium Citrate | 0.5 | Tetrahydrate | |
| Disodium Succinate | 0.1 | Copper Sulfate | 1.2 |
| Ammonium Sulfate | 0.5 | Ammonium Vanadate | 0.5 |
| Ammonium Hydrogen | 0.25 | Cobalt Sulfate | 0.5 |
| Carbonate | | Heptahydrate | |
| Potassium | 0.25 | Boric Acid | 0.6 |
| Dihydrogenphosphate | | Nickel Sulfate Hexahydrate | 0.5 |
| Magnesium Carbonate | 0.6 | Vitamin B₁ | 2.5 |
| Calcium Carbonate | 0.12 | Vitamin B₁₂ | 0.005 |

### 3. Plasmid DNA for Introducing Drug Resistance Gene

Plasmid DNA for introducing a drug resistance gene was prepared as described below.

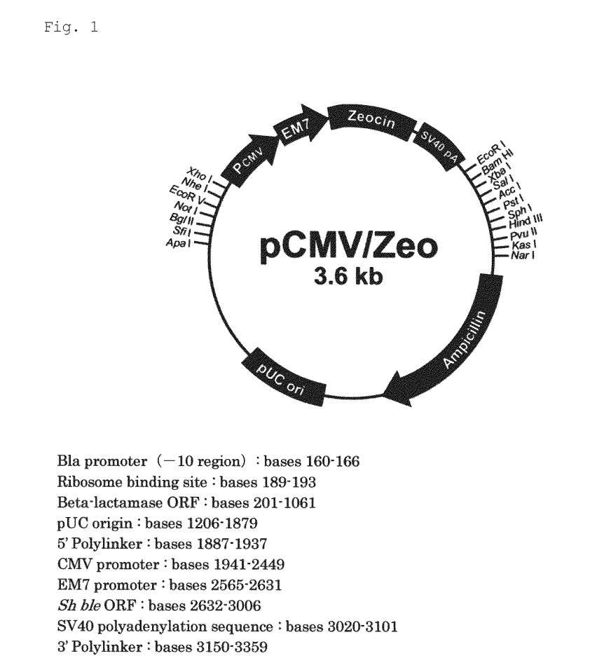

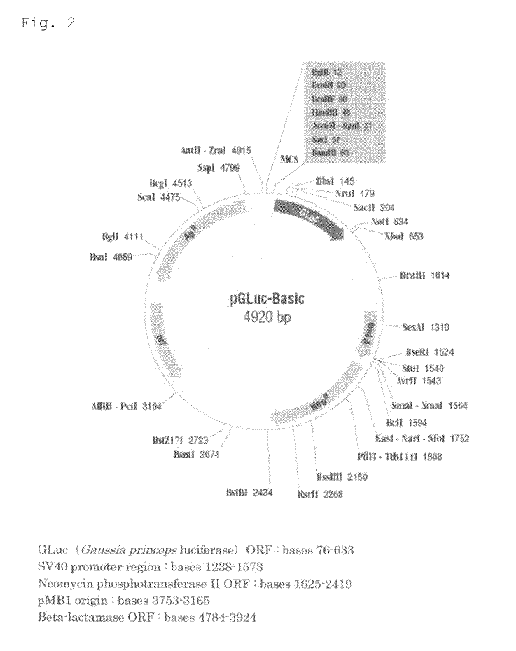

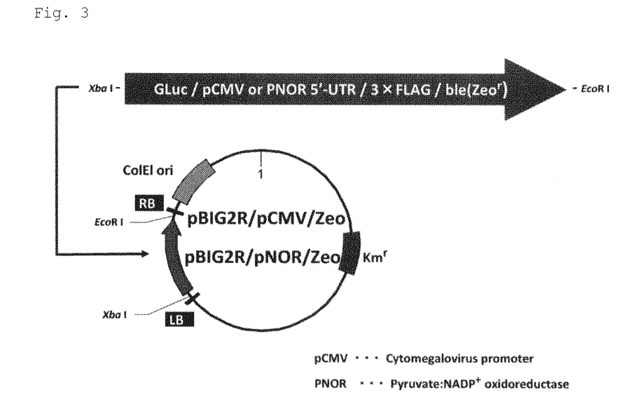

A drug resistance gene ble cassette inserted into the T-DNA region was prepared as follows. First, 3×FLAG, an epitope tag, was inserted into the 5′ end of the ble gene of pCMV/Zeo (Invitrogen, FIG. 1). Further, the EcoR I and BamH I sites were destroyed by blunting with Blunting High (Toyobo). The plasmid was cleaved with Not I and Xba I and inserted into pGLuc-Basic (New England Biolab, FIG. 2). The thus-obtained plasmid was cleaved with EcoR I and Xba I, thereby obtaining a ble cassette. The ble cassette was inserted into the T-DNA region of pBIG2RHPH2 (FIG. 3) (Tsuji, G. et al., “Agrobacterium tumefaciens-mediated transformation for random insertional mutagenesis in Colletotrichum lagenarium,” Journal of General Plant Pathology, 2003, 69, pp. 230-239). pBIG2RHPH2, which is a shuttle vector that is usable for preparation of a binary vector, is derived from pBIN19 (Frish, D. A. et al. “Complete sequence of the binary vector Bin19,” Plant Molecular Biology, 1995, 27(2), pp. 405-409) and has Agrobacterium recognition sequences, Left Border (LB) and Right Border (RB); the resulting plasmid was used (FIG. 3). All of the ligation reactions were carried out with Ligation High (Toyobo) at 16° C. pBIG2R/pCMV/Zeo is the plasmid in which exogenous CMV promoter (pCMV) and EM7 promoter (pEM7) linked together were used in the promoter site. pBIG2R/pNOR/Zeo is the plasmid in which the region from pCMV to pEM7 was replaced by PNOR 5′UTR (pNOR), a Euglena endogenous promoter. Each plasmid was transformed into Agrobacterium after confirmation of the sequence.

### 4. Method for Culturing Agrobacterium

Culture was performed using an LB liquid medium. A plate medium containing 1.5% (w/v) of agar powder was used when Agrobacterium was revived from a glycerol stock and when a transformant was selected. Irrespective of the type of medium, all of the LB media mentioned in the following description were used after rifampicin was added to each medium at a final concentration of 50 μg/ml. An LB medium to which, in addition to rifampicin, kanamycin was added at a final concentration of 100 μg/ml was used for culturing an Agrobacterium transformant.

### 5. Transformation of Agrobacterium

### 5.1 Preparation of Agrobacterium Competent Cells

Agrobacterium competent cells were prepared according to the method of Cold Spring Harbor (Detlef, W. et al., “Transformation of Agrobacterium Using Electroporation,” 2006, Cold Spring Harbor Protocols). Agrobacterium was streaked from a glycerol stock onto an LB plate medium and subjected to static culture (28° C.). A single colony that appeared after two to three days was cultured in 3 ml of LB medium. 2 ml of the culture solution that reached the stationary phase was added to 200 ml of LB medium, followed by culture with shaking at 180 rpm. The culture solution in which OD₅₅₀ was 0.5 to 1.0 was centrifuged (4,000×g, 4° C., 10 min), and the precipitate was washed with sterile water three times. The amount of sterile water was 200 ml in the first wash and 100 ml in the second wash and in the third wash. The washed precipitate was suspended in 2 ml of 10% glycerol. The suspension was dispensed into microcentrifugation tubes by 50 μl, frozen with liquid nitrogen, and preserved as Agrobacterium competent cells at −80° C.

### 5.2 Agrobacterium Transformation by Electroporation

0.5 μg of the DNA was added to the competent cells, and the mixture was placed into a cuvette (2-mm gap). Thereafter, electroporation was performed under the following conditions.

### Apparatus: BTM ECM600 Electro Cell Manipulator

### Voltage: 2.4 kV

### Resistance: 129Ω

Electric capacity: 50 μF
Plasmid DNA amount: 1 μg

The cells after the pulse were collected, and an LB liquid medium was added to the cells, followed by culture with shaking at 28° C. for 2 to 4 h. The cultured cells were then spread in an LB selective plate medium (100 μg/ml kanamycin, 50 μg/ml rifampicin) and subjected to static culture at 28° C. A single colony that appeared after three to five days was used as an Agrobacterium transformant.

### 5.3 Euglena Transformation (Co-Culture)

Euglena was cultured in a KH medium (pH of 6.8) for 4 to 5 days and, after cell counting, suspended in an IM liquid medium to 5.0×10⁶ cells/ml.

The Agrobacterium transformant was subjected to preculture in an LB medium. The resulting transformant was inoculated in an IM medium (pH of 5.3) (Tables 2 to 4) and cultured for about 10 to 15 hours. The cultured bacteria cells were collected by centrifugation (7,700×g, 20° C., 1 min) and suspended in the IM liquid medium so that OD₆₆₀ was 0.6.

1 ml of the Euglena suspended in the liquid medium and 1 ml of the Agrobacterium transformant suspended in the liquid medium were mixed (a total of 2 ml of culture solution), and acetosyringone was added at a final concentration of 100 μM, followed by co-culturing for 48 hours while gently stirring with a rotator (2.5×10⁶ cells/ml, OD₆₆₀=0.3). After the completion of the culture, 200 μl of the culture solution (0.5×10⁶ cells) or 200 μl of a 10-fold dilution of the culture solution (0.50×10⁶ cells) was cultured in a KH selective plate medium (zeocin 25 μg/ml, cefotaxime 100 μg/ml).

TABLE 2

| Components | Amount | |
|---|---|---|
| AB Solution 1 (Table 3) | 5 | ml |
| AB Solution 2 (Table 4) | 5 | ml |
| MES | 40 | mM |
| Glucose | 10 | mM |
| Glycerol | 0.50% | |
| Acetosyringone | 100 | μM |

TABLE 3

| | | |
|---|---|---|
| Dipotassium Hydrogenphosphate | 12 | g |
| Potassium Dihydrogenphosphate | 4 | g |

| | |
|---|---|
| H₂O | Fill up to 200 ml |

TABLE 4

| | | |
|---|---|---|
| Ammonium Chloride | 4 | g |
| Magnesium Sulfate Heptahydrate | 1.2 | g |
| Potassium Chloride | 0.6 | g |
| Calcium Chloride Dihydrate | 0.6 | g |
| Iron Sulfate Heptahydrate | 10 | mg |

| | |
|---|---|
| H₂O | Fill up to 200 ml |

### 5.4 Collection and Subculture of Euglena Transformant

Cells subjected to drug selection were collected as follows. Culture was performed on the selective plate medium at 27° C. for about 2 weeks, and lawn-like cells on the plate were suspended in 5 ml of sterile water to collect the cells. The cells in the cell suspension were counted, and the cells (0.5×10⁶ cells) were cultured again in a selective plate medium (25 μg/ml or 50 μg/ml of zeocin, 100 μg/ml of cefotaxime). After growth, the cells were suspended and cultured again in the selective plate medium. The initial number of cells in culture was reduced from 0.5×10⁶ cells in a stepwise manner with each passage. After several passages, a single colony formed on the selective plate medium was collected and cultured in 3 ml of selective liquid medium (50 μg/ml of zeocin, 50 μg/ml of cefotaxime), the cells that showed good growth were used as an isolated Euglena transformant.

### 5.5 Investigating the Stability of Introduced Trait in Euglena Transformant

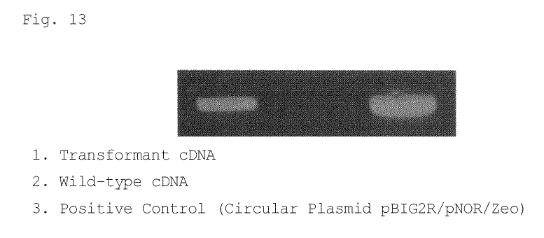

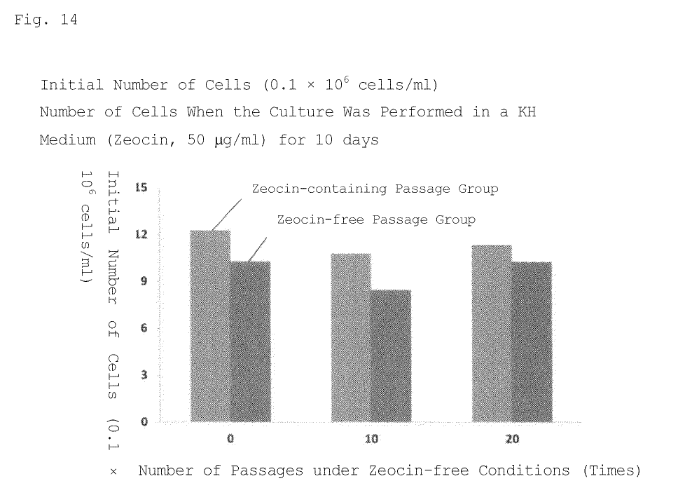

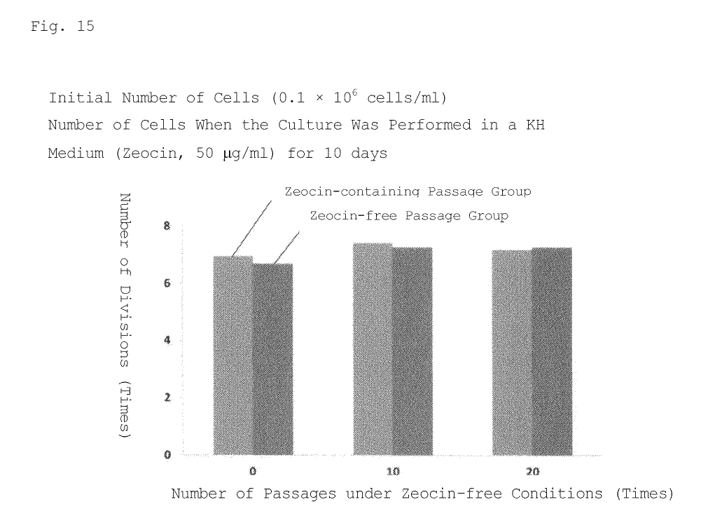

The cells were repeatedly subcultured in a KH liquid medium containing 50 μg/ml of zeocin, and the resulting cells were repeatedly subcultured in a zeocin-free KH liquid medium for the number of the times indicated in FIGS. 14 and 15. The subculturing was performed by transferring 1/150 the amount of cells into a fresh KH liquid medium every week.

The thus-obtained cells, which had not been exposed to the drug for a certain period of time, were cultured again in a KH liquid medium containing 50 μg/ml of zeocin, and their growth was investigated.

The cells that continued to be cultured in the absence of zeocin for a certain period of time were also used in the following DNA or RNA analysis.

### 5.6 Detection of Introduced Gene

5.6.1 Total DNA Extraction from Euglena Transformant

The Euglena transformant was cultured in a zeocin-containing medium or a zeocin-free medium for 4 to 5 days, collected by centrifugation, and washed twice with PBS(−) (the composition of 10×PBS(−) is shown in Table 5). The resulting transformant was suspended in NTES (Table 6) in an amount that was three to four times the volume of the cells and heated at 65° C. for 10 minutes. Further, the same amount of PCI was added, followed by vigorous stirring and centrifugation (17,400×g, 4° C., 5 min). To the supernatant collected from this, an equal amount of PCI was added, followed by stirring. Centrifugation was then performed again. To the collected supernatant, a 1/10-fold amount of 3M NaOAc was added. After inverting several times, a 2.5-fold amount of 100% ethanol was added, followed by mixing. The resulting mixture was allowed to stand at room temperature for 15 minutes and subjected to centrifugation (11,100×g, 4° C., 5 min). The supernatant was completely removed, and the precipitate was washed with 1 ml of 70% ethanol. The precipitate was air-dried at room temperature and dissolved in 20 μg/ml of a TE buffer containing RNase A, followed by RNA degradation at 37° C. overnight. The obtained solution was used as a total DNA solution.

TABLE 5

| | | |
|---|---|---|
| 80 | g | NaCl |
| 29 | g | Na₂HPO₄•12H₂O |
| 2 | g | KCl |
| 2 | g | KH₂PO₄ |

Fill up to 1 L

TABLE 6

| 0.1M | NaCl |
|---|---|
| 0.01M | Tris-HCl (pH of 7.5) |
| 1 mM | EDTA (pH of 8.0) |
| 1% | SDS |

### 5.6.2 PCR Using Total DNA

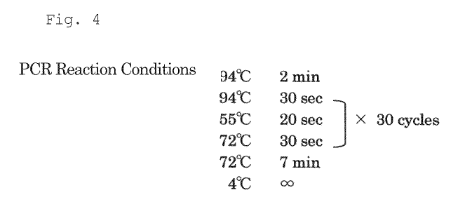

PCR was performed under the following conditions. Primers were designed so that they amplify the zeocin resistance gene region. Table 7 shows the PCR reaction system. FIG. 4 shows the PCR reaction conditions.

TABLE 7

| | | |
|---|---|---|
| 12.5 | μl | Go Taq Green Master Mix, |
| | | 2× (Promega) |
| 0.5 | μl | Forward Primer (20 μM) |
| 0.5 | μl | Reverse Primer (20 μM) |
| 0.5 | μl | DNA Solution |
| 11 | μl | Sterile Water |

The following PCR primers were used.

Forward Primer:

| | |
|---|---|
| 5′- ACCAGTGCCGTTCCGGTGCTCAC -3′ | (SEQ ID NO: 4) |

Reverse Primer:

| | |
|---|---|
| 5′- TGCTCGCCGATCTCGGTCATGG -3′ | (SEQ ID NO: 5) |

### 5.6.3 Agarose Gel Electrophoresis

Agarose was dissolved in TAE buffer (Table 8) so that the concentration was 1.5%, and ethidium bromide was added so that the concentration was 0.1 μg/ml, thereby preparing a gel. 5 μl of the reaction solution after the completion of PCR was applied to a well, and electrophoresis was performed at 100 V. DNA detection by UV irradiation was then performed using an AE-6905 (Arm).

TABLE 8

| | |
|---|---|
| 40 mM | Tris-acetate |
| 1 mM | EDTA |

### 5.7 Analysis of Transcription Product

5.7.1 Extraction of Total RNA from Euglena Transformant

ISOGEN II (NIPPON GENE) was used for the extraction of RNA, an RNase-free reagent was basically used as a reagent, and DEPC-treated water was used as water.

About 100 μl of the Euglena cells (volume) was collected and suspended by adding 1 ml of ISOGEN II. 400 μl of DEPC water was added thereto, and the mixture was vigorously stirred for 15 seconds and allowed to stand at room temperature for 15 minutes. After centrifugation (17,400×g, 4° C., 10 min) was performed, 1 ml of the supernatant was collected from the supernatant fraction, taking care not to take out portions near the precipitate. 1 ml of isopropanol was added thereto, and the mixture was mixed by inversion and then allowed to stand at room temperature for 10 minutes. After centrifugation (17,400×g, 4° C., 15 min), the supernatant was discarded, and 500 μl of 75% ethanol was added to the precipitate, followed by centrifugation (17,400×g, 4° C., 10 min). The precipitate was washed again with 75% ethanol and subjected to centrifugation (17,400×g, 4° C., 5 min). The supernatant was completely removed, and the precipitate was dissolved in 20 μl of DEPC water. The concentration of RNA was determined by measuring the A₂₆₀ value with a spectrophotometer.

### 5.7.2 RT-PCR

For a reverse transcription reaction, SuperScript II Reverse Transcriptase (Invitrogen) was used. RT-PCR was performed using 5 μg of the total RNA.

Reverse transcription reaction solution 1 with the composition shown in Table 9 was used.

TABLE 9

| | | |
|---|---|---|
| 1 | μl | (dt) ₁₇•AP Primer (100 μM) |
| x | μl (5 μg) | Total RNA Solution |
| 5 | μl | dNTP (2 mM) |
| 6 · x | μl | Sterile Water |

The following (dT)₁₇-AP primer was used.

(dT)₁₇-AP primer:
(SEQ ID NO: 6)
3′-GGCCACGCGTCGACTAGTACTTTTTTTTTTTTTTTTT -5′

Reaction solution 1 described above was incubated at 65° C. for 5 minutes and rapidly cooled on ice.

Reverse transcription reaction solution 2 with the composition shown in Table 10 was used.

TABLE 10

| | | |
|---|---|---|
| 12 | μl | Reaction Solution 1 after Rapid Cooling |
| 4 | μl | 5 × First-Strand Buffer |
| 2 | μl | DTT (0.1M) |
| 1 | μl | Toyobo RNase Inhibitor (40 U/μl) |

Reaction solution 2 described above was incubated at 42° C. for 2 minutes. 1 μl (200 units) of SuperScript II RT was added thereto, and a reverse transcription reaction was started. cDNA was thus obtained.

Conditions for the reverse transcription reaction was as shown in Table 11.

TABLE 11

| | |
|---|---|
| 42° C. | 50 min |
| 70° C. | 15 min |
| 4° C. | ∞ |

PCR was performed using the synthesized cDNA as a template and primers designed so that they detect the drug resistance gene ble.

The results are as described below.

### 6.1 Effect of Zeocin on Euglena Wild Strain in KH Medium at pH of 6.8

### 6.1.1 Minimum Inhibitory Concentration of Zeocin for Euglena Wild Strain

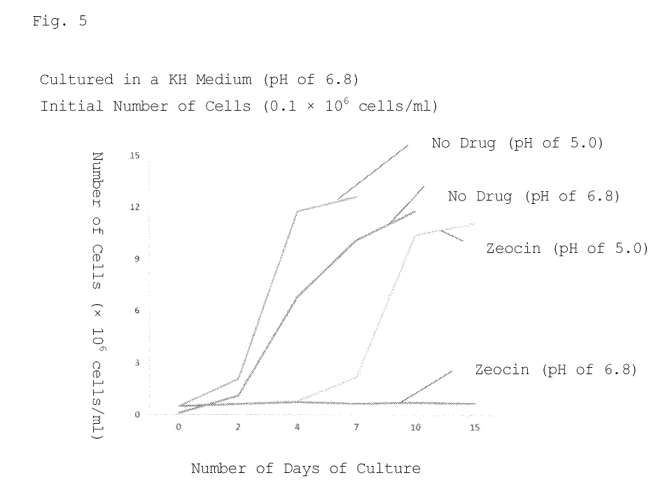

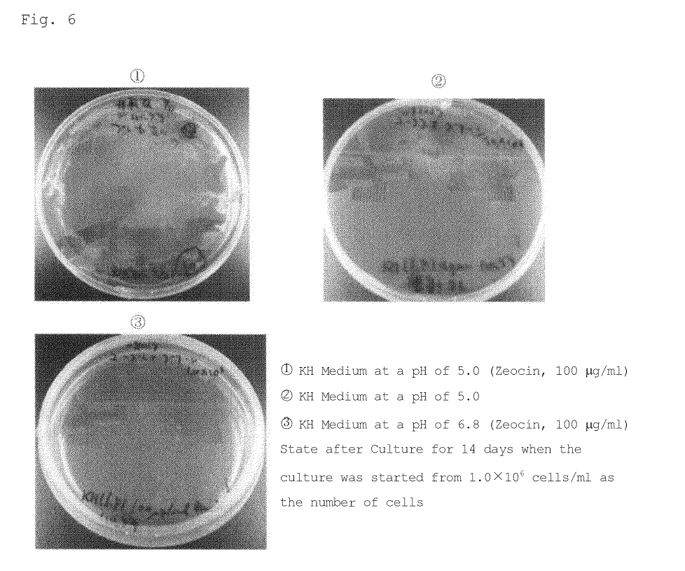

The minimum inhibitory concentration of zeocin for the Euglena wild strain in a medium at a pH of 6.8 was examined. The results reveal that in a liquid medium, the growth can be completely inhibited at 50 μg/ml, whereas in a plate medium, the growth can be nearly inhibited at 25 μg/ml. The growth inhibition was observed even when the number of cells at the start of culture was relatively large (2.0×10⁶ cells/ml in a liquid medium, 2.0×10⁶ cells/plate in a plate medium). In a medium containing 100 μg/ml of zeocin, the growth of the Euglena wild strain was almost completely inhibited, whether in a liquid medium (FIG. 5) or a plate medium (FIG. 6).

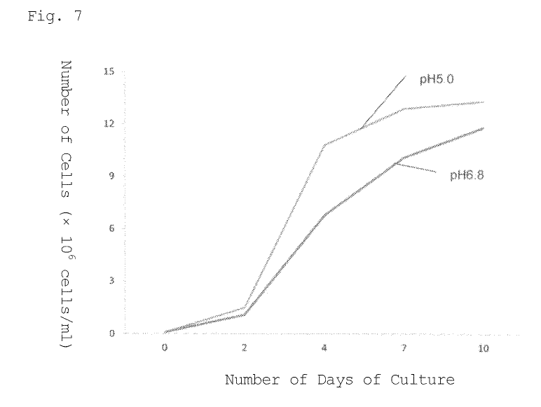

FIG. 7 shows the difference in the growth of the Euglena wild strain in a KH medium at each pH

### 6.1.2 Sensitivity of Euglena Wild Strain to Various Drugs

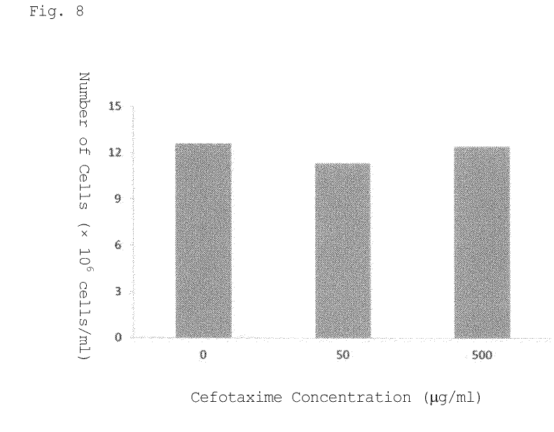

The influence of cefotaxime, which is used to remove Agrobacterium, on the growth of Euglena in transformation by the Agrobacterium method was examined. The results reveal that cefotaxime had no influence on the growth of the Euglena wild strain to at least 500 μg/ml (FIG. 8). Thus, a selective medium containing cefotaxime was used.

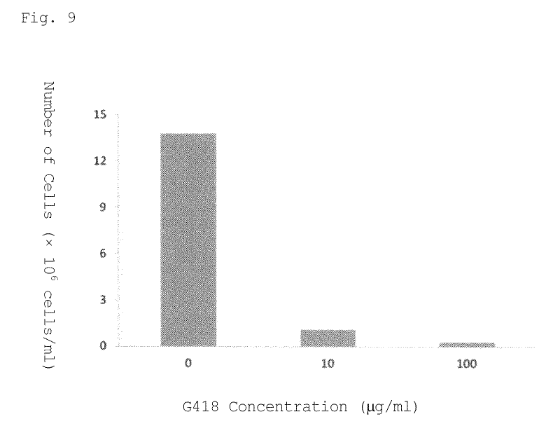

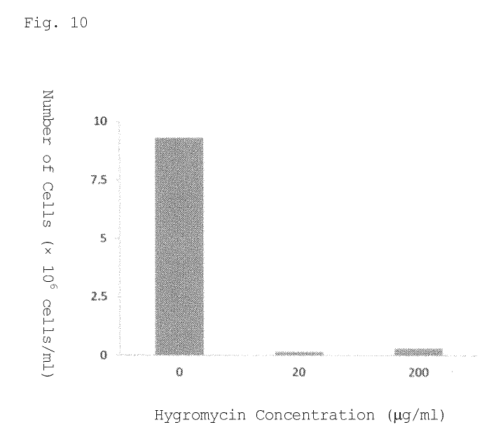

G418 and hygromycin inhibited the growth of the Euglena wild strain as the concentrations increased (FIGS. 9 and 10). This suggests the possibility that these drugs, in addition to zeocin, can be used as selection markers of the Euglena transformant.

### 6.1.3 Consideration of Conditions for Co-Culturing

Co-culturing was performed by the method described in Section 5.3. The liquid media used for co-culturing were a KH medium and an IM medium. An IM medium is frequently used when transformation by the Agrobacterium method is performed. The media used in the present experiment had a pH of 5.3, contained acetosyringone, which is an inducer of vir genes, at a concentration of 100 μM, and contained glucose, which is an inducing factor, at a concentration of 10 mM. The results of co-culturing using these media reveal that better growth of the Euglena transformant on a selective medium was observed when the IM medium was used. A comparison of the system using exogenous pCMV as an introduced gene promoter and the system using endogenous pNOR as an introduced gene promoter reveals that better growth tended to be shown in the system using pNOR.

### 6.1.4 Consideration of Selective Medium Conditions

After the completion of co-culturing, the culture solution was washed with cefotaxime (cefotaxime sodium, 500 μg/ml). In a selective medium, the zeocin concentration was set to 25 μg/ml, and the cefotaxime concentration was set to 100 μg/ml. The transformant was effectively obtained by increasing the zeocin concentration in a stepwise manner in isolation of the transformant.

### 6.2 Drug Resistance of Euglena Transformant

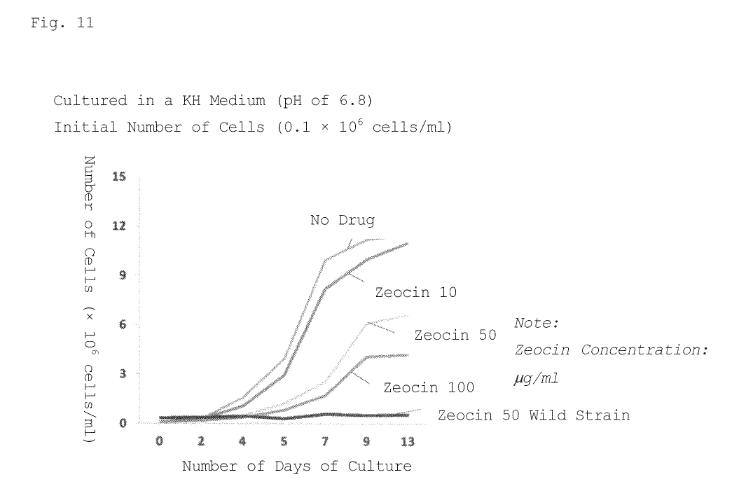

The drug resistance of the isolated Euglena transformant was observed. The Euglena transformant was cultured in a KH medium and a selective liquid medium (each with pH of 6.8). The results reveal that the transformant cells showed relatively good growth; however, there was a large difference from the growth in the conditions in which no drug was contained FIG. 11). In view of the results in Section 6.1.1, it is believed that almost no cells, except for the transformant, grow in the selective liquid medium at a pH of 6.8. Accordingly, the isolation of the transformant with the drug is considered to have been sufficient. More specifically, this suggests the possibility that the cause of insufficient drug resistance that the transformant showed is not imperfect isolation of the cell line. One of the causes for the insufficient drug resistance is a problem of the expression amount of the introduced gene. There is the possibility that a sufficient expression amount was not obtained with the promoters used in this experiment. Additionally, in the Agrobacterium method, although an introduced gene is inserted into the genome, the site is random. Since the expression amount of an introduced gene tends to depend on the site into which the introduced gene is inserted, there is the possibility that, depending on the site of insertion, the expression will not reach a sufficient level.

6.3 Detection of Introduced Gene from Euglena Transformant

Analysis by PCR was performed using the total DNA extracted from the Euglena transformant as a template. The fragment corresponding to the zeocin resistance gene region was successfully amplified (FIG. 12).

6.4 Analysis of Transcription Product Derived from Introduced Gene

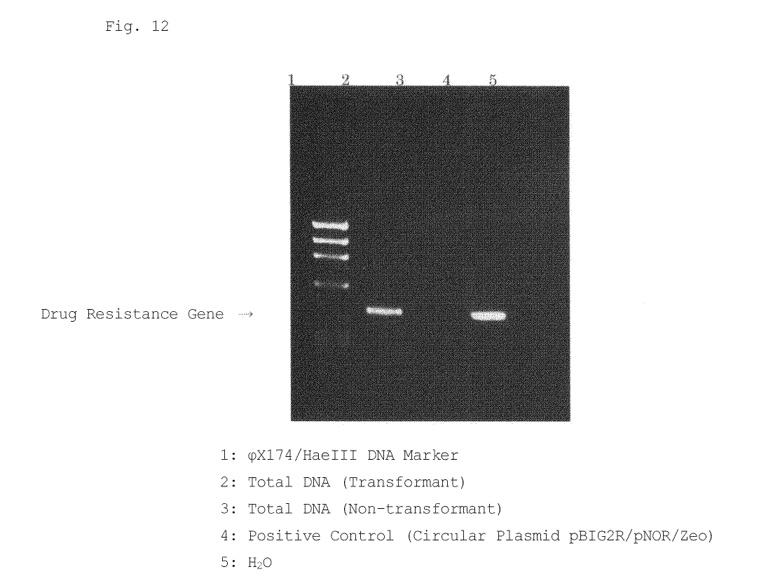

cDNA was synthesized using the total RNA extracted from the Euglena transformant as a template. Detection of the zeocin resistance gene was attempted using the cDNA as a template. The fragment corresponding to this was obtained (FIG. 13).

### 6.5 Stability of Introduced Gene

Passaging of the transformant was continued under zeocin-free conditions, and the resulting transformant was subcultured again in a zeocin-containing medium. A comparison of growth between these two reveals that sufficient growth was confirmed in both cases (FIG. 14). This indicates that drug resistance is sufficiently maintained even if culture is performed in the absence of the drug for a period of time. Additionally, a comparison of the number of divisions from the start of culture shows little difference between the two. Thus, the trait acquired by transformation is considered to be stably maintained (FIG. 15).

### 7. Selection and Analysis of Transformant by Using G418

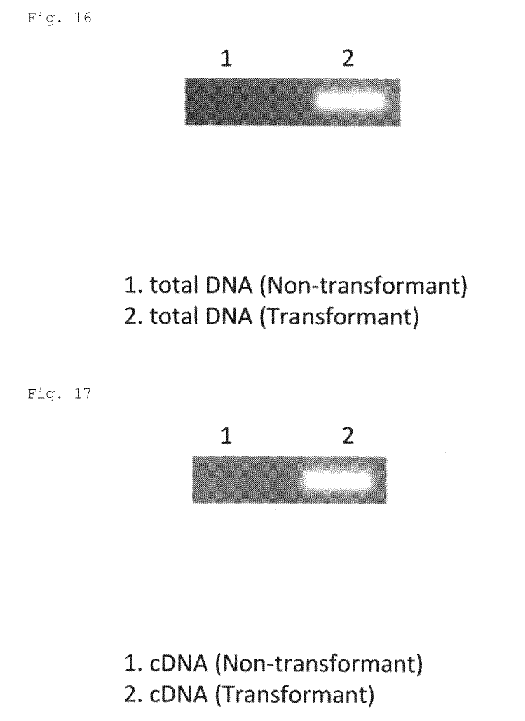

An experiment was performed introducing a neomycin resistance gene, which is responsible for G418 resistance. The neomycin resistance gene used is shown in SEQ ID NO: 7. The experiment was performed in the same manner as in the selection for zeocin. The experiment was performed at a G418 concentration of 10 μg/ml. As shown in FIGS. 16 and 17, DNA and transcription product of the neomycin resistance gene were detected from the obtained G418-resistant transformant.

### 8. Selection and Analysis of Transformant by Using Hygromycin

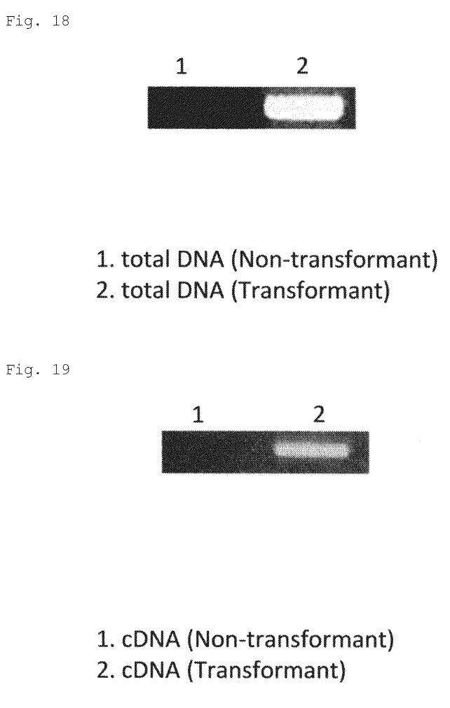

An experiment was performed introducing a hygromycin resistance gene. The hygromycin resistance gene used is shown in SEQ ID NO: 8. The experiment was performed in the same manner as in the selection for zeocin. The experiment was performed at a hygromycin concentration of 10 μg/ml. As shown in FIGS. 18 and 19, DNA and transcription product of the hygromycin resistance gene were detected from the obtained hygromycin-resistant transformant.

1. A Euglena carrying a drug resistance gene and a foreign gene of interest in an expressible manner.

2. The Euglena according to claim 1, which carries the drug resistance gene and the foreign gene of interest in an expressible manner until it is subcultured for at least 10 passages in the absence of the drug.

3. The Euglena according to claim 1, wherein the drug is zeocin, hygromycin, or G418.

4. The Euglena according to claim 1, which is obtainable by a method comprising the step of (1) introducing the drug resistance gene and the foreign gene of interest into a Euglena by the Agrobacterium method.

5. A method for producing a Euglena carrying a drug resistance gene and a foreign gene of interest in an expressible manner, the method comprising the step of:
(1) introducing the drug resistance gene and the foreign gene of interest into a Euglena by the Agrobacterium method.

6. The method according to claim 5, further comprising the step of:
(2) culturing the Euglena obtained in step (1) in the presence of the drug.

7. The method according to claim 5, wherein the drug is zeocin, hygromycin, or G418.

8. The method according to claim 7, wherein the culture is performed at a pH of 6 to 8.
